(19) United States
(12) Patent Application Publication
(10) Pub. No.: US 2024/0008565 A1
(43) Pub. Date: Jan. 11, 2024

(54) SCALED COMPOSITE STRUCTURE

(71) Applicant: NK Technology ltd., London (GB)

(72) Inventor: Natalie Carolin KERRES, (US)

(21) Appl. No.: 18/251,548

(22) PCT Filed: Nov. 18, 2021
(86) PCT No.: PCT/IB2021/060698

§ 371 Date: May 3, 2023

(30) Foreign Application Priority Data
Nov. 19, 2020 (GB) 2018186.3

(51) Int. Cl. A41D 13/015; G05B 19/4097; A41D 31/24

(52) U.S. Cl. CPC A41D 13/0153; G05B 19/4097; A41D 31/245; G05B 2219/35134

(57) ABSTRACT

A scaled composite structure (200) comprises a flexible base layer arrangement (204) and a plurality of three-dimensional (3D) scales (202A-N) attached to the flexible base layer arrangement. The plurality of 3D scales within the scaled composite structure overlap with each other when the scaled composite structure is placed on a planar surface. A range of motion of the scaled composite structure is controlled through a size and a shape of each of the plurality of 3D scales such that the plurality of 3D scales are intersecting with each other when a limit of the range of motion is applied to the scaled composite structure, to provide a mechanical interlocking effect.

### TECHNICAL FIELD

The present disclosure relates generally to a scaled composite structure; more specifically, the present disclosure relates to a method for designing and manufacturing a scaled composite structure based on at least one input parameter.

### BACKGROUND

In general, it is known to use protective structures to protect and support human or animal body parts from damage. For example, in the health care sector, protective structures are designed to support body parts whilst protecting the body parts from injury or damage. However, known protective structures usually have restricted movement, namely the known protective structures are often not sufficiently flexible. The known protective structures, as used in the health care sector, provide constant protection and support of body parts, which is potentially disadvantageous because the protective structures tend to reduce muscle strength in crucial body areas.

There has been ongoing research to develop a flexible protective structure that is capable of protecting as well as allowing a range of the motion to occur. The research may develop a protective structure that is made of flexible and thin material for providing impact protection to body parts.

Furthermore, research on animal scales reveals that the animal scales serve not only as protection against predators, but also address environmental challenges such as friction, compression or hyperextension.

One common drawback to the aforementioned known protective structures is that they restrict motion while providing protection.

Therefore, there arises a need to address the aforementioned technical drawbacks in existing technologies in developing protective structures.

### SUMMARY

The present disclosure seeks to provide an improved scaled composite structure. Moreover, the present disclosure seeks to provide a method for designing and manufacturing the scaled composite structure. An aim of the present disclosure is to provide a solution that overcomes at least partially the problems encountered in prior art.

According to a first aspect, there is provided a scaled composite structure, characterized in that the scaled composite structure comprises:
(i) a flexible base layer arrangement; and
(ii) a plurality of three-dimensional (3D) scales attached to the flexible base layer arrangement, wherein the plurality of 3D scales are overlapping when the scaled composite structure is placed on a planar surface,
wherein a range of motion of the scaled composite structure is controlled through a size and a shape of each of the plurality of 3D scales such that the plurality of 3D scales are intersecting with each other when a limit of the range of motion is applied to the scaled composite structure, to provide a mechanical interlocking effect.

The scaled composite structure is of advantage in that the scales provide a mechanical interlocking effect that is able to limit a range of motion of the scaled composite structure. Furthermore, each of the plurality of 3D scales provide mechanical protection against impact damage. According to a given limit of the range of the motion, the size and the shape of each of the plurality of 3D scales is beneficially customized.

Optionally, in the scaled composite structure, the flexible base layer arrangement enables each of the plurality of 3D scales to only rotate around a base plane in a centre point of each of the plurality of 3D scales.

Optionally, in the scaled composite structure, the flexible base layer arrangement operates against a force that is produced when the limit of the range of motion is applied to the scaled composite structure until interlocking of each of the plurality of 3D scales such that the scaled composite structure provides impact protection against produced force through force distribution.

Optionally, in the scaled composite structure, the size and the shape of at least a given scale of the plurality of 3D scales is a function of a location of the given scale within the scaled composite structure.

Optionally, in the scaled composite structure, the plurality of 3D scales change color when a force applied over the scaled composite structure exceeds a threshold value.

Optionally, in the scaled composite structure, a length and a width of each of the plurality of 3D scales are in a range from 0.01 millimetres (mm) to 500 mm.

Optionally, in the scaled composite structure, each of the plurality of 3D scales comprises a body portion and a nose portion. The nose portion may be characterized as a front extension of each of the plurality of 3D scales which overlaps a preceding 3D scale in the scaled composite structure.

Optionally, in the scaled composite structure, each 3D scale is attached to the flexible base layer arrangement via a plurality of teeth provided at a surface of the 3D scale facing towards the flexible base layer arrangement, wherein the plurality of teeth are arranged to penetrate through the flexible base layer arrangement to attach to a base plate.

According to a second aspect, there is provided a wearable protective device, characterized in that the wearable protective device comprises:
(i) a flexible base layer arrangement; and
(ii) a plurality of three-dimensional (3D) scales attached to the flexible base layer arrangement, wherein the plurality of 3D scales are overlapping when the wearable protective device is placed on a planar surface;
wherein a range of motion of the wearable protective device is controlled through a size and a shape of each of the plurality of 3D scales such that the plurality of 3D scales are intersecting with each other when a limit of the range of motion is applied to the scaled composite structure, to provide a mechanical interlocking effect.

The wearable protective device is of advantage in that the device prevents hyperextension injuries caused by sudden impact (example: collision or fall), prevents repetitive strain injury (RSI) through repetitive wrong motion, or limits the motion to help rehabilitation processes. The wearable protective device may be flexible in the range of motion and interlocking the motion when the limit of the range of motion is applied to the wearable protective device. The wearable protective device may also protect a body joint or a body part from impact through force distribution.

Optionally, the wearable protective device further comprises at least one of a double-sided adhesive layer, a type of hydrogel adhesive layer, a silicone adhesive layer or a rubber adhesive layer on one side of the wearable protective device or a sleeve that is interlaced with the wearable protective device for attaching to a skin of a wearer.

According to a third aspect, there is provided a method for designing and manufacturing a scaled composite structure, characterized in that the scaled composite structure comprises a plurality of three-dimensional (3D) scales that are attached to a flexible base layer arrangement, wherein the method comprises:
determining, by using a data processing arrangement, a flexible base layer arrangement and a shape of the flexible base layer arrangement, based on at least one input parameter, at a limit of a range of motion to be applied to the scaled composite structure;
determining, by using the data processing arrangement, a size and a shape of each of the plurality of 3D scales that are arranged on the
   flexible base layer arrangement when curved to the limit of the range of motion, thereby determining a length of each of the plurality of 3D scales; and
manufacturing the scaled composite structure based on the size and the shape of each of the plurality of 3D scales that are arranged on the flexible base layer arrangement when curved to the limit of the range of motion, such that the scaled composite structure provides a mechanical interlocking effect when the limit of the range of motion is applied to the scaled composite structure and is flexible until each of the plurality of 3D scales interlock with each other.

The method is of advantage in that it enables production of a series of mass-customized and flexible scaled composite structures for precise motion control, based on the at least one input parameter.

Optionally, in the method, the at least one input parameter is selected from at least one of a type of a part to be covered by the scaled composite structure, data associated with a range of motion of the part, physical parameters of the part, and a type of activity to be performed by the part or on the part. The method may be optimized for cost effectiveness, lightweight of the scaled composite structure and resisting potential of the scaled composite structure to applied force.

Optionally, the method further comprises:
determining, by using the data processing arrangement, an overall size of each of the plurality of 3D scales and a distance between each of the plurality of 3D scales, based on the at least one input parameter;
tessellating, by using the data processing arrangement, the flexible base layer arrangement when curved to the limit of the range of motion based on the overall size of the each of the plurality of 3D scales and the distance between each of the plurality of 3D scales; and
arranging, by using the data processing arrangement, the plurality of 3D scales on the flexible base layer arrangement according to a size and the shape of a base unit after tessellating the flexible base layer arrangement when curved to the limit of the range of motion to determine the size and the shape of each of the plurality of 3D scales.

Optionally, the method further comprises estimating a force to be applied to the scaled composite structure based on the at least one input parameter for enabling determining the overall size of each of the plurality of 3D scales and the distance between each of the plurality of 3D scales.

Optionally, the method further comprises determining (i) a thickness of each of the plurality of 3D scales; (ii) a type of the flexible base layer arrangement for connecting each of the plurality of 3D scales; and (iii) a material for manufacturing the plurality of 3D scales, based on estimated force.

Optionally, the method further comprises determining a diameter and a height of teeth like structures in each of the plurality of 3D scales based on a degree of fineness and a thickness of the type of the first layer, wherein the plurality of teeth are configured to penetrate through the flexible base layer arrangement to attach the 3D scales to the flexible base layer arrangement.

Optionally, the method further comprises determining an intersection point between each two scales of the plurality of scales in a row of scales disposed on the flexible base layer arrangement when curved to the limit of the range of motion, to determine the length of each of the plurality of scales in the flexible base layer arrangement when curved to the limit of the range of motion.

Optionally, the method further comprises generating a 3D model of the scaled composite structure based on a flattened flexible base layer arrangement with the plurality of 3D scales for enabling manufacturing of the scaled composite structure.

Optionally, in the method, the size and the shape of at least one a given scale of the plurality of 3D scales are a function of a location of the given scale within the scaled composite structure. The mechanical interlocking effect of the scaled composite structure is controlled through the size and the shape of each of the plurality of 3D scales.

Optionally, the manufacturing of the scaled composite structure comprises
providing a flexible base layer arrangement;
generating a bottom layer of at least one scale of the plurality of 3D scale;
optionally generating a plurality of teeth like structures projecting from the bottom layer;
adding a first layer above the bottom layer; and
generating a top layer of the at least one scale of the plurality of 3D scales on top of the first layer.

Optionally, the manufacturing of the scaled composite structure comprises connecting the plurality of 3D scales together using at least one of: a base plate, living hinges, stiches.

Optionally, the plurality of 3D scales are arranged radially or linearly.

Optionally, the plurality of 3D scales are imprinted with one or more sensors.

According to a fourth aspect, there is provided a computer program product comprising instructions to carry out the method of the third aspect.

It will be appreciated that the aforesaid present method is not merely “software for a computer, as such”, “methods of doing a mental act, as such”, but has a technical effect in that the method includes designing the scaled composite structure, using the data processing arrangement, in a distributed computing architecture and manufacturing the scaled composite structure using a scale forming unit based on a design of the scaled composite structure. The method for designing the scaled composite structure uses at least one of a parametric algorithm or a non-parametric algorithm to address, for example to solve, the technical problem of designing the scaled composite structure. The present disclosure works as a combination of software and hardware for designing and manufacturing the scaled composite structure.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system for designing and manufacturing a scaled composite structure, in accordance with an embodiment of the present disclosure;

FIG. 2A is a top view of an exemplary scaled composite structure in a bent position, in accordance with an embodiment of the present disclosure;

FIG. 2B is a side view of a three-dimensional (3D) scale of FIG. 2A that has teeth-like structures in accordance with an embodiment of the present disclosure;

FIG. 3A is a front view of an exemplary three-dimensional (3D) scale, in accordance with an embodiment of the present disclosure;

FIG. 3B is a side view of an exemplary three-dimensional (3D) scale, in accordance with an embodiment of the present disclosure;

FIG. 3C is a top view of an exemplary three-dimensional (3D) scale, in accordance with an embodiment of the present disclosure;

FIG. 4 is an illustration of a network surface of an exemplary three-dimensional (3D) scale's half body portion, in accordance with an embodiment of the present disclosure;

FIG. 5A is a side view of a first three-dimensional (3D) scale and a second 3D scale in a row in a planar surface, in accordance with an embodiment of the present disclosure;

FIG. 5B is a side view of a first three-dimensional (3D) scale and a second 3D scale in a row in a YZ-plane, in accordance with an embodiment of the present disclosure;

FIG. 6 is an illustration of an exemplary curved surface arranged with a plurality of three dimensional (3D) scales, in accordance with an embodiment of the present disclosure;

FIG. 7A is a vector diagram that illustrates a relationship of a first three-dimensional (3D) scale and a second 3D scale in a first position in a YZ-plane, in accordance with an embodiment of the present disclosure;

FIG. 7B is a vector diagram that illustrates a relationship of a first three-dimensional (3D) scale and a second 3D scale in a second position in a YZ-plane, in accordance with an embodiment of the present disclosure;

FIG. 8A is a top view of an exemplary wearable protective device, in accordance with an embodiment of the present disclosure;

FIG. 8B is a side view of a three-dimensional (3D) scale of FIG. 8A that has teeth-like structures in accordance with an embodiment of the present disclosure;

FIG. 9 is an exemplary graphical representation of a prediction of an injury based on information retrieved from one or more sensors embedded in a scaled composite structure, in accordance with an embodiment of the present disclosure;

FIG. 10 is a flowchart illustrating steps of a method for designing and manufacturing a scaled composite structure, in accordance with an embodiment of the present disclosure;

FIG. 11 is a schematic diagram of an exemplary method for designing a scaled composite structure for protecting a body joint, in accordance with an embodiment of the present disclosure;

FIG. 12 is a schematic diagram of an exemplary method for manufacturing a scaled composite structure, in accordance with an embodiment of the present disclosure;

FIGS. 13A-13B are flow charts of an exemplary method for designing and manufacturing a scaled composite structure for protecting a body joint, in accordance with an embodiment of the present disclosure;

FIG. 14 is an illustration of an exploded view of a distributed computing architecture or a system in accordance with an embodiment of the present disclosure;

FIGS. 15A and 15B are perspective views of the plurality of 3D scales connected through a base plate in accordance with an embodiment of the present disclosure;

FIGS. 16A, 16B and 16C are perspective views of the plurality of 3D scales connected through stitches in accordance with an embodiment of the present disclosure;

FIGS. 17A and 17B are perspective views of the plurality of 3D scales connected through living hinges in accordance with an embodiment of the present disclosure;

FIGS. 18A, 18B and 18C are illustrations of exemplary living hinges for connecting plurality of 3D scales in accordance with an embodiment of the present disclosure; and

FIG. 19 is an illustration of an exemplary scaled composite structure in accordance with various embodiments of the present disclosure.

### DETAILED DESCRIPTION OF EMBODIMENTS

The following detailed description illustrates embodiments of the present disclosure and ways in which they can be implemented.

According to a first aspect, there is provided a scaled composite structure, characterized in that the scaled composite structure comprises:
(i) a flexible base layer arrangement; and
(ii) a plurality of three-dimensional (3D) scales attached to the flexible base layer arrangement, wherein the plurality of 3D scales are overlapping when the scaled composite structure is placed on a planar surface,
wherein a range of motion of the scaled composite structure is controlled through a size and a shape of each of the plurality of 3D scales such that the plurality of 3D scales are intersecting with each other when a limit of the range of motion is applied to the scaled composite structure, to provide a mechanical interlocking effect.

The advantage of the scaled composite structure is that it provides the mechanical interlocking effect when the limit of the range of the motion is applied to the scaled composite structure. Otherwise, the scaled composite structure is flexible in the range of the motion. Furthermore, the size and the shape of each of the plurality of 3D scales are a function of the mechanical interlocking effect of the scaled composite structure. According to a given limit of the range of the motion, the size and the shape of each of the plurality of 3D scales are customized. The plurality of 3D scales are flexible in one direction and interlock in another direction.

For example, the scaled composite structure may be used as a motion limiting structure for a wearer to prevent hyperextension of any body joint or a body part, thereby the scaled composite structure prevents injuries of the body joint or the body part. According to parameters associated with the wearer such as a body weight, a body height, a type of the body joint or the body part to be protected, a type of activity (example: rehabilitation, sports performance enhancement, protection, hyperextension protection, impact protection), anthropometry, an age, and medical conditions of the wearer (example: osteoarthritis, carpal tunnel syndrome, etc.), the size and the shape of each of the plurality of 3D scales are controlled to provide the mechanical interlocking effect when the limit of the range of the motion is reached by the body joint or the body part.

Furthermore, the scaled composite structure provides protection to the body joint or the body part without reducing muscle strength of the wearer. The scaled composite structure provides protection to the body joint or the body part when the limit of the range of the motion is reached by the body joint or the body part. Otherwise, the scaled composite structure remains flexible in the range of the motion. The range of the motion may represent a healthy range of motion of the body joint or the body part.

The scaled composite structure may prevent hyperextension injuries caused by sudden impact (example: collision or fall), repetitive strain injury (RSI) through repetitive wrong motion, or limits the motion to help a rehabilitation process.

The scaled composite structure may be used to protect cables from damage or prevent over-flexing of the cables. The scaled composite structure may be used to protect cables that have application in robotics and automobiles. The cables may be armoured cables. The scaled composite structure may be used as a firefighting cloth or a bullet-proof vest.

The flexible base layer arrangement includes a first layer that may be at least one of a fabric or flexible layer; for example, the fabric is a woven fabric or a unitary layer with perforations. A layer of printed plastics material, for example Nylon®, polyamide material, polypropylene material or similar, may be added between a bottom layer and a top layer of each of the plurality of 3D scales. Alternatively, the fabric or flexible layer can be positioned beneath the bottom layer of each of the plurality of 3D scales which may be connected to the top layer. Alternatively, the fabric or flexible layer can be positioned above the top layer of each of the plurality of 3D scales which may be connected to the bottom layer.

The thickness of the first layer may be in a range of 0.05 mm to 2 mm, more optionally in a range of 0.1 mm to 1 mm, namely greater than or equal to 0.1 millimetre (mm) for example. The thickness of the first layer may be equivalent to one layer of a 3D scale.

Optionally, the flexible base layer arrangement has a Young's Modulus in the range from 0.2 to 10 Megapascals (MPa), and has a tensile strength in the range from 0.5 to 20 MPa. In a layman's terms, the flexible base layer arrangement has a Young's modulus and tensile strength generally similar to a woven fabric used for clothing articles.

Optionally, in the scaled composite structure, the flexible base layer arrangement enables each of the plurality of 3D scales to only rotate around a base plane in a centre point of each of the plurality of 3D scales.

Optionally, in the scaled composite structure, the flexible base layer arrangement operates against a force that is produced when the limit of the range of motion is applied to the scaled composite structure until interlocking of each of the plurality of 3D scales such that the scaled composite structure provides impact protection against produced force through force distribution.

When the range of motion is applied to the scaled composite structure, the force may be created in each of the plurality of 3D scales. Thus, it leads to substantial force distribution, not only on the scaled composite structure itself but also on the flexible base layer arrangement that is connecting each of the plurality of 3D scales. During the range of motion, the flexible base layer arrangement operates against created force by enabling each of the plurality of 3D scales to only rotate in the centre point of each of the plurality of 3D scales, thereby the scaled composite structure provides the impact protection against the force through force distribution. The scaled composite structure may provide protection against collision and friction. Moreover, optionally, the plurality of 3D scales are arranged radially or linearly. It will be appreciated that a radially arranged plurality of 3D scales enables better force distribution in comparison to a linearly arranged plurality of 3D scales. Therefore, the radially arranged plurality of 3D scales may be useful on uneven or surfaces that are rounded or not flat. In an example, the radially arranged plurality of 3D scales may be used in applications, such as kneepads, elbow pads, and the like.

Optionally, in the scaled composite structure, the size and the shape of at least a given scale of the plurality of 3D scales is a function of a location of the given scale within the scaled composite structure. For example, a length of a 3D scale positioned on a flat area is higher than to a length of a 3D scale that is positioned on a curved area. Moreover, the radially arranged plurality of 3D scales in the scaled composite structure may control motion in 2 axes, such as angle of rotation in x-y plane. It will be appreciated that for the plurality of 3D scales to be able to control angle of rotation, the plurality of 3D scales is arranged on (namely, attached to) a flexible base layer arrangement, rather than printed flat.

In an embodiment, at least a portion of the plurality of 3D scales in the scaled composite structure are mutually different in the size, and the shape. The at least a portion of the plurality of 3D scales in the scaled composite structure may have an identical size and shape.

Optionally, in the scaled composite structure, the plurality of 3D scales change colour (US: “color”) when a force applied over the scaled composite structure exceeds a threshold value.

For example, the plurality of 3D scales change color when the body joint or the body part is injured. The scaled composite structure may provide feedback to the wearer by changing the color of the plurality of 3D scales when the wearer met an accident or the force applied over the scaled composite structure exceeds the threshold value.

Optionally, in the scaled composite structure, a length and a width of the each of the plurality of 3D scales are in a range from 0.01 millimetres (mm) to 500 mm.

Optionally, in the scaled composite structure, each of the plurality of 3D scales comprises a body portion and a nose portion. The nose portion may be characterized as a front extension of each of the plurality of 3D scales which overlaps a preceding 3D scale in the scaled composite structure. A length of the nose portion of the plurality of 3D scales may determine the range of motion on which each of the plurality of 3D scales to rotate until the plurality of 3D scales interlock.

In an embodiment, a material that is used for producing the plurality of 3D scales is selected from a group comprising any resin, carbon, carbon fibre, aramid fibre, Polylactic acid (PLA) polyester, non-Newtonian silicon, Nylon, Polypropylene, Polycarbonate, Thermoplastic Polyurethane (TPU), Polyamide 11 (PA11), Polyamide 12 (PA12), steel, aluminum, elastomeric polyurethane (EPU 40), Rigid Polyurethane 70 (RPU 70), Urethane Methacrylate (UMA90), Acrylonitrile butadiene styrene (ABS), Polyvinyl Alcohol (PVA), Polycarbonate like Translucent, acrylate-based plastic, and ultra-tough white plastic.

Optionally, the material for producing the plurality of 3D scales is selected from a group comprising of polyetheretherketone (PEEK), polyurethane (PU), ethylene vinyl acetate (EVA), polystyrene (PS), styrene acrylonitrile (SAN), acrylonitrile styrene acrylate (ASA), polyethylene terephthalate (PET), polymethyl methacrylate (PMMA), polytetrafluoroethylene (PTFE), Polyoxymethylene (POM), thermoplastic elastomer (TPE), rubber, plastic. Optionally, the material for producing the plurality of 3D scales includes ceramic.

In an embodiment, the first layer is selected from a group comprising of an aramid fibre, carbon fibre, glass fibre, hemp fibre, Nylon and polymer fibre.

The scaled composite structure may include one or more sensors to monitor the motion of the body joint or the body part for predicting injury that is going to happen. The one or more sensors may sense the injury if it happens to a wearer, to provide feedback to cure the injury. The one or more sensors may include at least one of an accelerator sensor, a gyroscope sensor, a flex sensor, an image sensor, a temperature sensor, a radiation sensor, a proximity sensor, a pressure sensor, an optical sensor, or a position sensor. The one or more sensors may be embedded in the scaled composite structure.

Optionally, the plurality of 3D scales are imprinted with the one or more sensors. In this regard, at least one of the plurality of 3D scales, that does not serve as a sensor or that avoids stress so that the parts are not damaged, is printed with at least one motherboard feature. The at least one motherboard feature has a plurality of split features that are printed on to the surrounding 3D scales. It will be appreciated that the electrical connections between the at least one motherboard feature and the plurality of split features is through conductive threads. Beneficially, the at least one motherboard feature and the plurality of split features are miniaturized arrangements that can be freely bent, wound, folded, moved, stretched and dynamically arranged in a three-dimensional space according to various space layout requirements. Additionally, the at least one motherboard feature and the plurality of split features can greatly reduce the volume and the weight of the scaled composite structure. In an embodiment, the at least one motherboard feature is implemented as a flexible printed circuit board (PCB).

According to a second aspect, there is provided a wearable protective device, characterized in that the wearable protective device comprises:
a flexible base layer arrangement; and
(ii) a plurality of three-dimensional (3D) scales attached to the flexible base layer arrangement, wherein the plurality of 3D scales are overlapping when the wearable protective device is placed on a planar surface;
wherein a range of motion of the wearable protective device is controlled through a size and a shape of each of the plurality of 3D scales such that the plurality of 3D scales are intersecting with each other when a limit of the range of motion is applied to the wearable protective device, to provide a mechanical interlocking effect.

The advantage of the wearable protective device is that it provides the mechanical interlocking effect when the limit of the range of the motion is applied to the wearable protective device. Furthermore, the size and the shape of each of the plurality of 3D scales that constitute the wearable protective device are a function of the mechanical interlocking effect of the wearable protective device. According to a given limit of the range of the motion, the size and the shape of each of the plurality of 3D scales is customized.

The wearable protective device may be used to prevent hyperextension of a body joint or a body part, thereby the wearable protective device prevents injuries of the body joint or the body part due to hyperextension.

The wearable protective device may also provide impact protection to a wearer. The wearable protective device may be flexible in the range of the motion.

Optionally, the wearable protective device further includes at least one of a double-sided adhesive layer, a type of hydrogel adhesive layer, a silicone adhesive layer or a rubber adhesive layer on one side of the wearable protective device or a sleeve that is interlaced with the wearable protective device for attaching to a skin of a wearer.

The wearable protective device may be used to prevent hyperextension injuries caused by sudden impact (example: collision or fall), repetitive strain injury (RSI) through repetitive wrong motion, or limit the motion to help rehabilitation process. The wearable protective device may be flexible and protect the body joint or the body part from impact.

According to a third aspect, there is provided a method for designing and manufacturing a scaled composite structure, characterized in that the scaled composite structure comprises a plurality of three-dimensional (3D) scales that are attached to a flexible base layer arrangement, wherein the method comprises:
determining, by using a data processing arrangement, a flexible base layer arrangement and a shape of the flexible base layer arrangement, based on at least one input parameter, at a limit of a range of motion to be applied to the scaled composite structure;
determining, by using the data processing arrangement, a size and a shape of each of the plurality of 3D scales that are arranged on the flexible base layer arrangement when curved to the limit of the range of motion, thereby determining a length of each of the plurality of 3D scales; and
manufacturing the scaled composite structure based on the size and the shape of each of the plurality of 3D scales that are arranged on the flexible base layer arrangement when curved to the limit of the range of motion, such that the scaled composite structure provides a mechanical interlocking effect when the limit of the range of motion is applied to the scaled composite structure and is flexible until each of the plurality of 3D scales interlock with each other.

The method is of advantage in that it enables production of a series of mass-customized and flexible scaled composite structures for precise motion control, based on the at least one input parameter. The method includes controlling the mechanical interlocking effect of the scaled composite structure through the size and a shape of each of the plurality of 3D scales such that the plurality of 3D scales are overlapping when the scaled composite structure is placed on a planar surface and are intersecting with each other when the limit of the range of motion is applied to the scaled composite structure.

In an embodiment, the data processing arrangement employs at least one of a parametric algorithm or a non-parametric algorithm to design the size and shape of the each of the plurality of 3D scales based on the at least one input parameter. The parametric algorithm or the non-parametric algorithm may be a machine learning algorithm, a regression algorithm, an artificial intelligence (AI) algorithm, or a neural network algorithm.

The method may be used to design and manufacture the scaled composite structure for protecting a body joint or a body part. The method may be used to design and manufacture a firefighting cloth or a bullet-proof vest. The method may be used to design and manufacture the scaled composite structure for protecting cables.

In an embodiment, a shape of the flexible base layer arrangement is determined at a limit of its requirement movement, using the data processing arrangement, based on a two-dimensional curve that is determined based on the limit of the range of motion, a length of the scaled composite structure needed and a width of the scaled composite structure needed.

In an embodiment, the two-dimensional curve is determined using at least one of an image processing model, an artificial intelligence (AI) model, an anthropometry statistics-based model, a 3D camera or a depth camera based on the at least one input parameter. The two-dimensional curve is determined, based on an image of a part to be covered in the limit of the range of the motion, using the at least one of the image processing model, the artificial intelligence (AI) model, the anthropometry statistics-based model, the 3D camera or the depth camera. The image of the part may be a three-dimensional (3D) image or a two-dimensional (2D) image.

The flexible base layer arrangement represents the limit of the range of the motion and covers at least a given area of a part. The part may be the body part or the body joint. The limit of the range of the motion may be a maximum degree of the motion.

A shape of a base unit may include at least one of a rectangular shape, a diamond shape, a rhombus shape, a square shape, a hexagon shape, or a circular shape. The shape of the base unit represents the base shape of each of the plurality of 3D scales.

Optionally, in the method, the at least one input parameter is selected from at least one of a type of a part to be covered by the scaled composite structure, data associated with a range of motion of the part, physical parameters of the part, and a type of activity to be performed by the part or on the part.

In an embodiment, the data associated with the range of motion of the part may include at least one of the image of part to be covered in the limit of the range of motion, or a manual input that includes the limit of the range of motion.

The at least one input parameter may be selected from a type of the body joint or the body part to be protected, an image of the body joint or the body part in the limit of the range of the motion, the limit of the range of the motion, a length and a width of the scaled composite structure needed to cover the body joint or the body part, a body weight, a body height, the type of activity to be performed by a wearer (rehabilitation, sports performance enhancement, protection, hyperextension protection, impact protection), maximum weight of the scaled composite structure, local anthropometry, an age of the wearer, medical conditions of the wearer (osteoarthritis, carpal tunnel syndrome, etc.), color preference of the scaled composite structure, and a choice of whether the scaled composite structure will be worn over or under clothing.

Optionally, the method further includes:
determining, by using the data processing arrangement, an overall size of each of the plurality of 3D scales and a distance between each of the plurality of 3D scales, based on the at least one input parameter;
tessellating, by using the data processing arrangement, the flexible base layer arrangement when curved to the limit of the range of motion based on the overall size of the each of the plurality of 3D scales and the distance between each of the plurality of 3D scales; and
arranging, by using the data processing arrangement, the plurality of 3D scales on the flexible base layer arrangement according to a size and the shape of the base unit after tessellating the flexible base layer arrangement when curved to the limit of the range of motion to determine the size and the shape of each of the plurality of 3D scales.

A centre point of each base unit in the tessellated flexible base layer arrangement when curved to the limit of the range of motion may represent a centre point of each of the plurality of the 3D scale's base.

In an embodiment, arranging the plurality of 3D scales on the flexible base layer arrangement when curved to the limit of the range of motion includes moving a body portion of each of the plurality of 3D scales to the centre point of each base unit. Normal vectors in the centre point of each base unit may be used to choose a direction of each of the plurality of 3D scales. A length of the normal vectors describes a size of the body portion of the plurality of 3D scales. Only the body portion of the plurality of 3D scales may be moved on the centre point of each base unit and, in a next step, the length of each of the plurality of 3D scales are determined to define a nose portion of each of the plurality of 3D scales.

Optionally, the method further includes estimating a force to be applied to the scaled composite structure based on the at least one input parameter for enabling determining the overall size of each of the plurality of 3D scales and the distance between each of the plurality of 3D scales.

The method may include further estimating the force to be applied to the scaled composite structure, based on the physical parameters of the part, and the type of activity to be performed by the part or on the part. The method may include estimating the force to be applied to the scaled composite structure by the wearer or the body joint or body part, based on the body weight, the body height, and the type of activity such as the rehabilitation, the sports performance enhancement, the protection, the hyperextension protection, and the impact protection.

In an embodiment, the method further includes determining a density of the scale composite structure, a base size of each of the plurality of 3D scales, based on an estimated force to be applied by the part or the wearer. The method may include further determining the distance between each of the plurality of 3D scales based on the density of the scale composite structure. The method may include further determining the overall size of each of the plurality of 3D scales based on the base size of each of the plurality of 3D scales.

Optionally, the method further comprises determining (i) a thickness of each of the plurality of 3D scales; (ii) a type of a first layer of the flexible base layer arrangement for connecting each of the plurality of 3D scales; and (iii) a material for manufacturing the plurality of 3D scales, based on estimated force.

Optionally, the method further comprises determining a diameter and a height of teeth-like structures in each of the plurality of 3D scales based on a degree of fineness and a thickness of the type of the flexible base layer arrangement, wherein the teeth-like structures are configured to penetrate through the flexible base layer arrangement to attach the 3D scales thereto.

The method may include determining a degree of fineness of the flexible base layer arrangement and a thickness of the flexible base layer arrangement, based on the type of the flexible base layer arrangement.

Optionally, the method further includes determining an intersection point between each two scales of the plurality of 3D scales in a row of 3D scales disposed on the flexible base layer arrangement when curved to the limit of the range of motion, to determine the length of each of the plurality of 3D scales in the flexible base layer arrangement.

The intersection point may be calculated using a following equation (1)

$$y = \frac{\begin{matrix}( h - {a}_{1} \times sin α - {a}_{2} \times cos α ) \times \\ ( cos α ( {a}_{1} - {b}_{1} ) + \\ sin α ( h - {a}_{2} )\end{matrix}}{\begin{matrix}( sin α ( {a}_{1} - {b}_{1} ) + \\ cos α ( {a}_{2 \cdot } - h )\end{matrix}} + ( {a}_{1} \times cos α - {a}_{2 \cdot } \times sin α ) \qquad equation ( 1 )$$

Optionally, the method further includes generating a 3D model of the scaled composite structure based on a flattened flexible base layer arrangement with the plurality of 3D scales for enabling manufacturing of the scaled composite structure. During the flattening process, the tessellated flexible base layer arrangement arranged with the plurality of 3D scales, a location, and size and shape of each of the plurality of 3D scales remain constant. Each of the plurality of 3D scales do not touch each other in the planar surface.

In an embodiment, the method further includes developing a geometric code (GCODE) based on the 3D model of the scaled composite structure for enabling manufacturing of the scaled composite structure.

Optionally, in the method, the size and the shape of at least one a given scale of the plurality of 3D scales are a function of a location of the given scale within the scaled composite structure. The mechanical interlocking effect of the scaled composite structure is controlled through the size and the shape of each of the plurality of 3D scales.

Optionally, the manufacturing of the scaled composite structure includes
providing a flexible base layer arrangement;
generating a bottom layer of at least one scale of the plurality of 3D scales;
optionally generating a plurality of teeth like structures projecting from the bottom layer;
adding a first layer above the bottom layer; and
generating a top layer of the at least one scale of the plurality of 3D scales on top of the first layer.

In an embodiment, the manufacturing of the scaled composite structure includes
moulding the top layer of the at least one scale of the plurality of 3D scales along with or without the plurality of teeth like structures as a one-step moulding process;
adding the first layer below the top layer, wherein the teeth like structures optionally penetrate through the first layer; and
connecting the bottom layer to the top layer by 3D printing or by thermal fuse welding.

The bottom layer and the top layer of the at least one scale of the plurality of 3D scales are optionally connected with a one-way pin system when moulded parts of the bottom layer and the top layer are put together.

The one-step moulding process may include at least one of an injection moulding, or an over-moulding. A method of manufacturing the scaled composite structure includes at least one of an additive manufacturing or 3D printing, or moulding.

In an embodiment, the method includes positioning the first layer in between the bottom layer and the top layer of at least one scale of the plurality of 3D scales. Alternatively, the first layer can be positioned beneath the bottom layer which may be connected to the top layer. Alternatively, the first layer can be positioned above the top layer which may be connected to the bottom layer.

In an embodiment, the method further includes attaching the scaled composite structure onto a skin of the wearer through at least one of: a double-sided adhesive, a type of hydrogel adhesive layer, a silicone adhesive layer or a rubber adhesive layer included on the scaled composite structure, or a sleeve that is interlaced with the scaled composite structure.

In an embodiment, the method further includes monitoring, using one or more sensors, the part covered with the scaled composite structure, and predicting the injury to the part covered with the scaled composite structure based on the data retrieved from the one or more sensors. The one or more sensors may be embedded in the scaled composite structure. The one or more sensors may include at least one of an accelerator sensor, a gyroscope sensor, a flex sensor, an image sensor, a temperature sensor, a radiation sensor, a proximity sensor, a pressure sensor, an optical sensor, or a position sensor.

The method may include further providing feedback to cure injury of the part if the injury happens to the part, based on the data retrieved from the one or more sensors.

The method for designing and manufacturing the scaled composite structure for protecting the body part may include (i) receiving at least one input parameter that includes data associated with the body part to be protected, the body specifications of the wearer, and the type of activity to be performed by the wearer, (ii) determining a flexible base layer arrangement that represents the limit of the motion and covers at least a portion of the body part and a shape of the flexible base layer arrangement, based on the data associated with the body part to be protected, (iii) determining an overall size of each of a plurality of 3D scales to be used for producing the scaled composite structure and a distance between each of the plurality of 3D scales, based on the body specifications of the wearer, and the type of activity to be performed by the wearer, (iv) tessellating the flexible base layer arrangement when curved to the limit of the range of motion based on the overall size of the each of the plurality of 3D scales and the distance between each of the plurality of 3D scales, (v) arranging the plurality of 3D scales on the tessellated flexible base layer arrangement according to a size and the shape of a base unit, (vi) determining a length of each of the plurality of 3D scales according to a location of each of the plurality of 3D scales in the flexible base layer arrangement to define a size and a shape of each of the plurality of 3D scales arranged on the flexible base layer arrangement, and (vii) generating the plurality of 3D scales that are connected together by the flexible base layer arrangement to produce the scaled composite structure, based on the size and the shape of each of the plurality of 3D scales arranged on the flexible base layer arrangement.

In an embodiment, there is provided a system for designing and manufacturing a scaled composite structure. The system includes a memory that stores a set of instructions, a data processing arrangement that is in communication with the memory, and a scale forming unit. When in operation, the data processing arrangement is configured to execute the set of instructions to perform (i) receiving at least one input parameter from a user, (ii) determining a flexible base layer arrangement and a shape of the flexible base layer arrangement, based on at least one input parameter, at a limit of a range of motion to be applied to the scaled composite structure (iii) determining a size and a shape of each of a plurality of 3D scales that are arranged on the flexible base layer arrangement when curved to the limit of the range of motion, thereby determining a length of each of the plurality of 3D scales, and (iv) enabling the scale forming unit to manufacture the scaled composite structure. The scale forming unit manufactures the scaled composite structure based on the size and the shape of each of the plurality of 3D scales that are arranged on the flexible base layer arrangement when curved to the limit of the range of motion. The data processing arrangement may be communicatively connected with the scale forming unit. The scale forming unit may be a three-dimensional (3D) printer, or a moulding apparatus.

Optionally, the manufacturing of the scaled composite structure comprises connecting the plurality of 3D scales together, wherein the plurality of 3D scales are connected together using at least one of: a base plate, living hinges, or stiches. In this regard, each of the plurality of 3D scales are connected together for allowing a range of motion of the scaled composite structure. Beneficially, connecting each of the plurality of 3D scales together results in a high strength, flexible scaled composite structure.

Optionally, the plurality of 3D scales that are arranged on the flexible base layer arrangement are connected using a base plate method. In the base plate method, a base plate is 3D printed. The base plate comprises a plurality of holes therein to reduce stress points. Subsequently, the plurality of 3D scales are printed on top of the base plate. Optionally, 3D printing of the base plate and the plurality of 3D scales on top of the base plate is done by SLS printing. Notably, SLS printing allows adjusting material quantities during printing. Beneficially, changing material of base plate during printing enables the printing of a stronger, more flexible scaled composite structure. It will be appreciated that while connecting the plurality of 3D scales using the base plate method the 3D printing process is not required to be paused before printing the plurality of 3D scales on top of the base plate.

Optionally, the plurality of 3D scales that are arranged on the flexible base layer arrangement are connected using a living hinges method. In the living hinges method, the plurality of 3D scales are 3D printed together based on the pre-determined size and structure of the wearable protective device. The plurality of 3D scales are connected together though living hinges without requiring a base plate. Beneficially, the use of living hinges results in a stronger, more flexible scaled composite structure. It will be appreciated that while connecting the plurality of 3D scales using the living hinges method the 3D printing process is not required to be paused before printing the plurality of 3D scales and connecting the plurality of 3D scales though living hinges.

Optionally, the plurality of 3D scales that are arranged on the flexible base layer arrangement are connected using a stitch method. More optionally, the plurality of 3D scales are connected using Kevlar stiches. The Kevlar (also called para-aramid) is a heat-resistant and strong synthetic fiber that is typically spun into ropes or fabric sheets that can be used as such, or as an ingredient in composite material components. Herein, the Kevlar stitches are used to bond the base unit of a scale and top part of an adjacent 3D scale. Beneficially, said manufacturing using Kevlar stitches provides better adhesion between the 3D scales and the Kevlar. Moreover, connecting the plurality of 3D scales using Kevlar stiches results in a stronger, more flexible, cheaper and more environmentally friendly scaled composite structure. Additionally, while connecting the plurality of 3D scales using Kevlar stiches, a lesser amount of Kevlar is required. It will be appreciated that while connecting the plurality of 3D scales using Kevlar stiches the 3D printing process is required to be paused while incorporating each Kevlar stitch between the base unit of a scale and the top part of an adjacent 3D scale.

The data processing arrangement and scale forming unit may be located remotely and may work as cohesive units. The data processing arrangement may communicate with the scale forming unit over a network. The network may be a wired network, e.g., fiber optic, Ethernet, Fiber Channel, direct connections, and close-range communications, a wireless network, e.g., Wi-Fi, combination of the wired network and the wireless network, a LAN (local area network), WAN (wide area network), the Internet, cable networks, and cellular networks.

The system may include at least one input interface to receive the at least one input parameter and at least one output interface to provide information about the size and the shape of each of the plurality of 3D scales.

The data processing arrangement may include one or more processors to execute the set of instructions stored in the memory and a database that stores a data driven model to perform one or more functions of the data processing arrangement.

Embodiments of the present disclosure substantially eliminate or at least partially address the aforementioned technical drawbacks in existing technologies in controlling motion by providing the scaled composite structure that interlocks when the limit of the range of the motion is applied to the scaled composite structure, otherwise the scaled composite structure remains flexible.

### DETAILED DESCRIPTION OF THE DRAWINGS

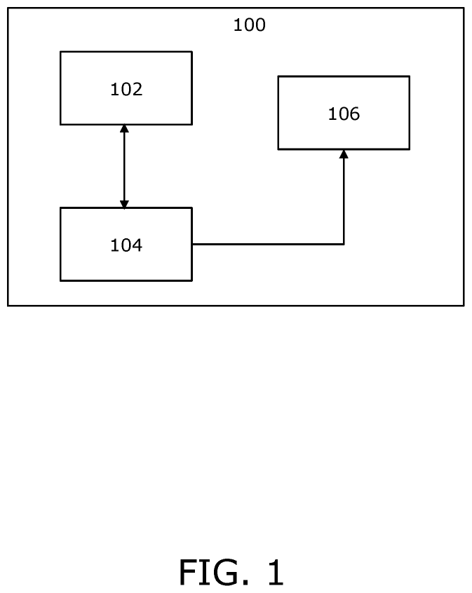

FIG. 1 is a schematic illustration of a system 100 for designing and manufacturing a scaled composite structure, in accordance with an embodiment of the present disclosure. The system 100 includes a memory 102 that stores a set of instructions, a data processing arrangement 104 that is in communication with the memory 102, and a scale forming unit 106. When in operation, the data processing arrangement 104 is configured to execute the set of instructions to perform (i) receiving at least one input parameter from a user or a wearer, (ii) determining a flexible base layer arrangement and a shape of the flexible base layer arrangement, based on at least one input parameter, at a limit of a range of motion to be applied to the scaled composite structure (iii) determining a size and a shape of each of a plurality of 3D scales that are arranged on the flexible base layer arrangement when curved to the limit of the range of motion, thereby determining a length of each of the plurality of 3D scales, and (iv) enabling the scale forming unit 106 to manufacture the scaled composite structure. The scale forming unit 106 manufactures the scaled composite structure based on the size and the shape of each of the plurality of 3D scales that are arranged on the flexible base layer arrangement when curved to the limit of the range of motion. The scaled composite structure provides a mechanical interlocking effect when a limit of a range of motion is applied to the scaled composite structure and is flexible in the range of the motion.

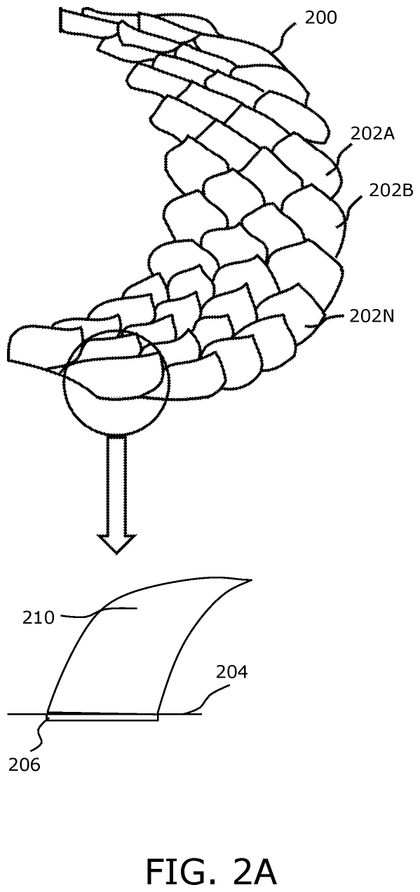

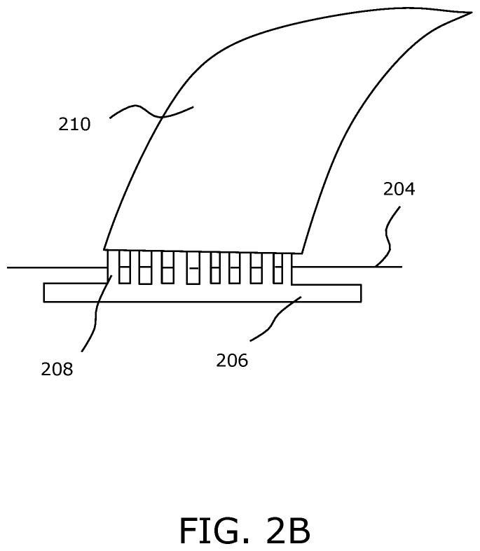

FIG. 2 is a top view of an exemplary scaled composite structure 200 in a bended position, in accordance with an embodiment of the present disclosure. The exemplary scaled composite structure 200 includes a plurality of three dimensional (3D) scales 202A-N and a flexible base layer arrangement 204. The plurality of three dimensional (3D) scales 202A-N are attached to the flexible base layer arrangement 204. Each of the plurality of 3D scales 202A-N includes a bottom layer 206 and a top layer 210 that is present on top of the flexible base layer arrangement 204. The plurality of 3D scales 202A-N are overlapping when the exemplary scaled composite structure 200 is placed on a planar surface. The plurality of 3D scales 202A-N are intersecting with each other when a limit of a range of motion is applied to the exemplary scaled composite structure 200, to provide a mechanical interlocking effect. The flexible base layer arrangement 204 may enable each of the plurality of 3D scales 202A-N to only rotate around a base plane in a centre point of each of the plurality of 3D scales 202A-N.

FIG. 2B is a side view of a three-dimensional (3D) scale of FIG. 2A that has a plurality of teeth 208 in accordance with an embodiment of the present disclosure. Each of the plurality of 3D scales 202A-N includes the bottom layer 206, a plurality of teeth 208, and the top layer 210 that is present on top of the plurality of teeth 208. The row of teeth-like structures 208 may penetrate through the flexible base layer arrangement 204.

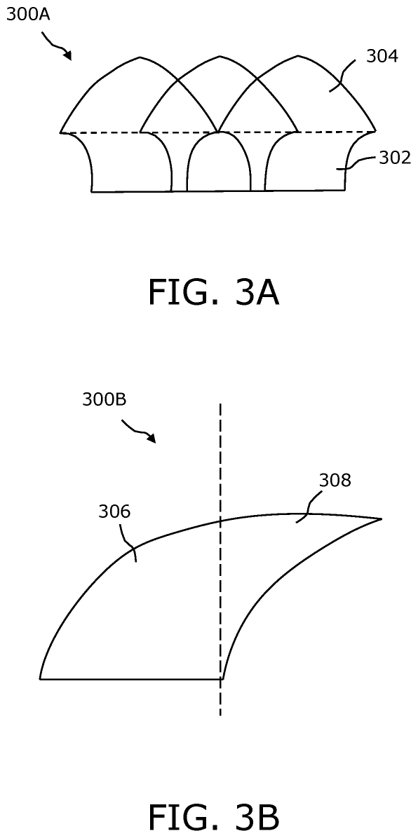

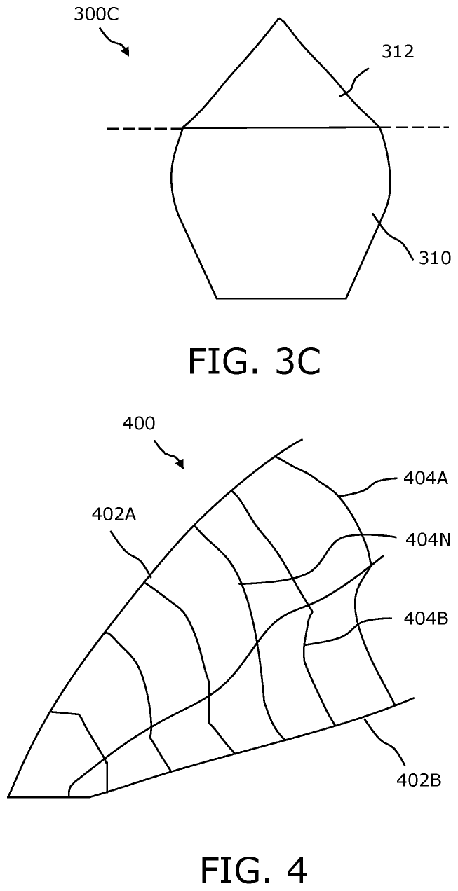

FIG. 3A is a front view of an exemplary three-dimensional (3D) scale 300A, in accordance with an embodiment of the present disclosure. The exemplary 3D scale 300A includes a body portion 302 and a nose portion 304. The body portion 302 is defined through a shape of a base unit of a flexible base layer arrangement that is determined based on at least one input parameter. The nose portion 304 is characterized as a front extension of the exemplary 3D scale 300A which overlaps a preceding 3D scale in a scaled composite structure. A length of the exemplary 3D scale 300A within the scaled composite structure may be controlled through given input parameters. Therefore, controlling the length of the exemplary 3D scale 300A allows the scaled composite structure to interlock when a limit of a range of motion is applied to the scaled composite structure.

FIG. 3B is a side view of an exemplary three-dimensional (3D) scale 300B, in accordance with an embodiment of the present disclosure. The exemplary 3D scale 300B includes a body portion 306 and a nose portion 308. A length of the nose portion 308 of the exemplary 3D scale 300B may determine how much the exemplary 3D scale 300B in front may be able to rotate until the exemplary 3D scale 300B interlock. The length of the nose portion 308 of the exemplary 3D scale 300B is controlled through given input parameters.

FIG. 3C is a top view of an exemplary three-dimensional (3D) scale 300C, in accordance with an embodiment of the present disclosure. The exemplary 3D scale 300C includes a body portion 310 and a nose portion 312. A length of the nose portion 312 of the exemplary 3D scale 300C controls flexibility of the exemplary 3D scale 300C in a scaled composite structure. Therefore, a nose portion of each 3D scale in the scaled composite structure has a specific length, depending on each 3D scale's location on the scaled composite structure.

FIG. 4 is an illustration of a network surface of an exemplary three-dimensional (3D) scale's half body portion 400, in accordance with an embodiment of the present disclosure. One or more first curves 402A-B in V direction and one or more second curves 404A-N in U direction are used to shape the exemplary 3D scale's half body portion 400.

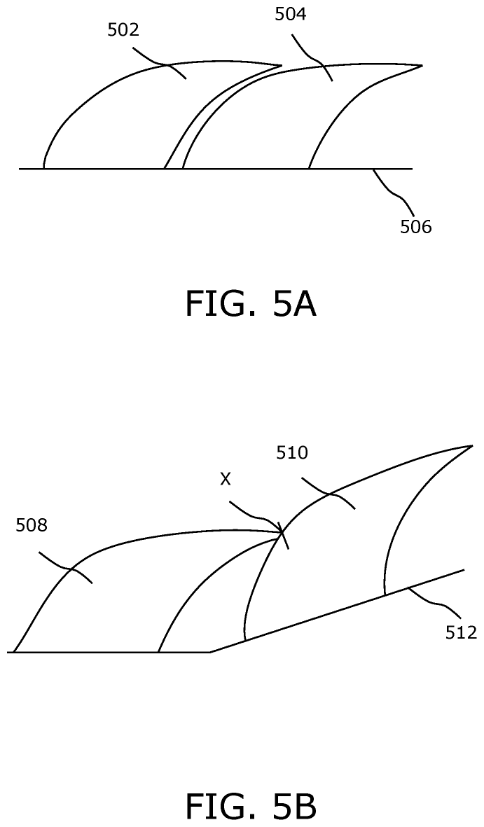

FIG. 5A is a side view of a first three-dimensional (3D) scale 502 and a second 3D scale 504 in a row in a planar surface 506, in accordance with an embodiment of the present disclosure. The first 3D scale 502 and the second 3D scale 504 do not intersect with each other (as shown in FIG. 5A) when the first 3D scale 502 and the second 3D scale 504 are placed on the planar surface 506. A base of the first 3D scale 502 and the second 3D scale 504 may be touching with each other. A size and a shape of the first 3D scale 502 and the second 3D scale 504 in a scaled composite structure are controlled through at least one input parameter. The size and the shape of the first 3D scale 502 and the second 3D scale 504 are a function of a location of the first 3D scale 502 and the second 3D scale 504 within the scaled composite structure.

FIG. 5B is a side view of a first three-dimensional (3D) scale 508 and a second 3D scale 510 in a row in a YZ plane 512, in accordance with an embodiment of the present disclosure. The first 3D scale 508 and the second 3D scale 510 are intersecting at an intersection point X (as shown in FIG. 5B) when the first 3D scale 508 and the second 3D scale 510 are placed on the YZ plane 512. The intersection point X may be calculated using a following equation (1):

$$y = \frac{\begin{matrix}( h - {a}_{1} \times sin α - {a}_{2} \times cos α ) \times \\ ( cos α ( {a}_{1} - {b}_{1} ) + \\ sin α ( h - {a}_{2} )\end{matrix}}{\begin{matrix}( sin α ( {a}_{1} - {b}_{1} ) + \\ cos α ( {a}_{2 \cdot } - h )\end{matrix}} + ( {a}_{1} \times cos α - {a}_{2 \cdot } \times sin α ) \qquad equation ( 1 )$$

The equation (1) may be used to determine a size and a shape of the first 3D scale 508 and the second 3D scale 510 within a scaled composite structure. The size and the shape of the first 3D scale 508 and the second 3D scale 510 are a function of a location of the first 3D scale 508 and the second 3D scale 510 within the scaled composite structure.

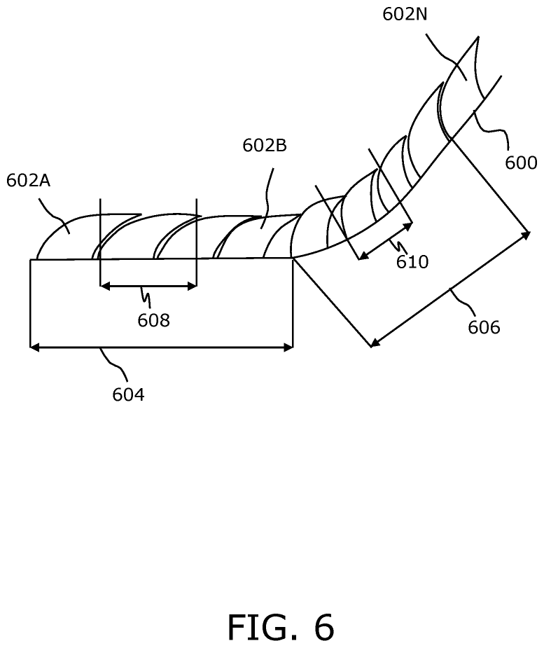

FIG. 6 illustrates an exemplary curved surface 600 arranged with a plurality of three dimensional (3D) scales 602A-N, in accordance with an embodiment of the present disclosure. The exemplary curved surface 600 is arranged with the plurality of 3D scales 602A-N. The exemplary curved surface 600 includes a flat area 604 and a curved area 606. Each of the plurality of three dimensional (3D) scales 602A-N has different length according to its location in the exemplary curved surface 600. For example, a 3D scale positioned in the flat area 604 has higher length 608 than a 3D scale positioned in the curved area 606. The 3D scale positioned in the curved area 606 has lower length 610 than the 3D scale positioned in the flat area 604. To calculate the length of each of the plurality of 3D scales 602A-N, an intersecting point of each two 3D scales at a rotation angle has to be calculated.

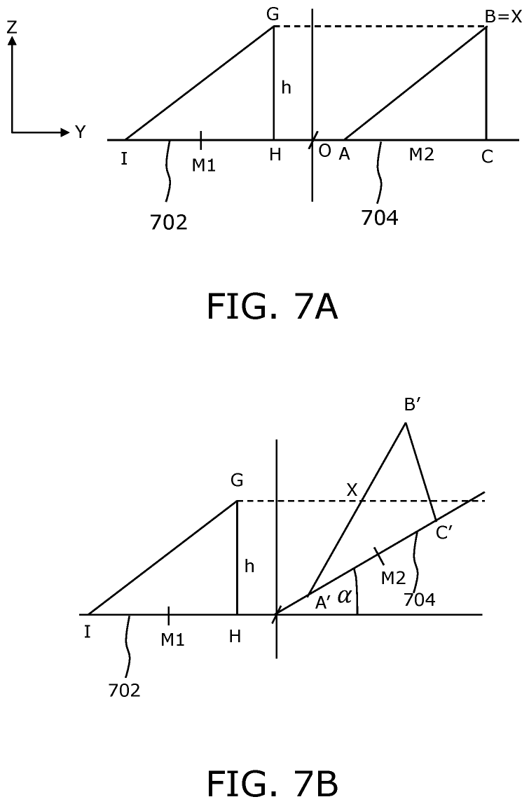

FIG. 7A is a vector diagram that illustrates a relationship of a first three-dimensional (3D) scale 702 and a second 3D scale 704 in a first position in YZ-plane, in accordance with an embodiment of the present disclosure. In FIG. 7A, to simplify a complex shape of an individual 3D scale, triangles represent the individual 3D scale's contour. The first 3D scale 702 and the second 3D scale 704 are placed on a planar surface in the first position. A point I represents a starting point of the first 3D scale 702. A point H represents an ending point of the first 3D scale 702. A point G represents a front point of a nose portion of the first 3D scale 702. The point G is parallel to their base as a vector from I to H. A point M1 represents a center point of the first 3D scale's 702 base shape.

Moreover, h represents a height of the first 3D scale 702. A point A represents a starting point of the second 3D scale 704. A point C represents an ending point of the second 3D scale 704. A point B represents a front point of a nose portion of the second 3D scale 704. The point B is parallel to their base as a vector from A to C. A point M2 represents a center point of the second 3D scale's 704 base shape. A point O represents a point of rotation between the first 3D scale 702 and the second 3D scale 704, which is a midpoint between the point H and the point A. Base planes of the first 3D scale 702 and the second 3D scale 704 are 0 degree rotated to one another. Therefore, a front point X is an intersection of the vector IH in the point G and a line between the points A and B. In the first position, the intersecting point X is equivalent to the point B.

FIG. 7B is a vector diagram that illustrates a relationship of a first three-dimensional (3D) scale 702 and a second 3D scale 704 in a second position in YZ-plane, in accordance with an embodiment of the present disclosure. In FIG. 7B, to simplify a complex shape of an individual 3D scale, triangles represent the individual 3D scale's contour. A point I represents a starting point of the first 3D scale 702. A point H represents an ending point of the first 3D scale 702. A point G represents a front point of a nose portion of the first 3D scale 702. A point M1 represents a center point of the first 3D scale's 702 base shape. h represents a height of the first 3D scale 702. A point A′ represents a starting point of the second 3D scale 704. A point C′ represents an ending point of the second 3D scale 704. A point B′ represents a front point of a nose portion of the second 3D scale 704. A point M2 represents a center point of the second 3D scale's 704 base shape. A point O represents a point of rotation between the first 3D scale 702 and the second 3D scale 704. In the second position, the first 3D scale 702 and the second 3D scale 704 are rotated around the point O with an angle α. A point X is an intersection point that moves along vector AB in the second Position. As the point X also remains on the vector IH in the point G, the intersection point X always has same height h and therefore an equivalent Z-coordinate as the point G. Vector calculations in two dimensional are applied to locate a Y-coordinate of the intersection point X (y z). A straight line is created through the points A′ and B′. For the straight line, equation y=kx+d, a slope k is calculated for the points A′ and B′ and consequentially d (intercept on a y-axis). The z-coordinate of the intersection point X is deployed. A following final equation (2) uses the points A and B from the first position (from FIG. 7A) and calculates the y-coordinate of the intersection point in the second position.

$$y = \frac{\begin{matrix}( h - {a}_{1} \times sin α - {a}_{2} \times cos α ) \times \\ ( cos α ( {a}_{1} - {b}_{1} ) + \\ sin α ( h - {a}_{2} )\end{matrix}}{\begin{matrix}( sin α ( {a}_{1} - {b}_{1} ) + \\ cos α ( {a}_{2 \cdot } - h )\end{matrix}} + ( {a}_{1} \times cos α - {a}_{2 \cdot } \times sin α ) \qquad equation ( 2 )$$

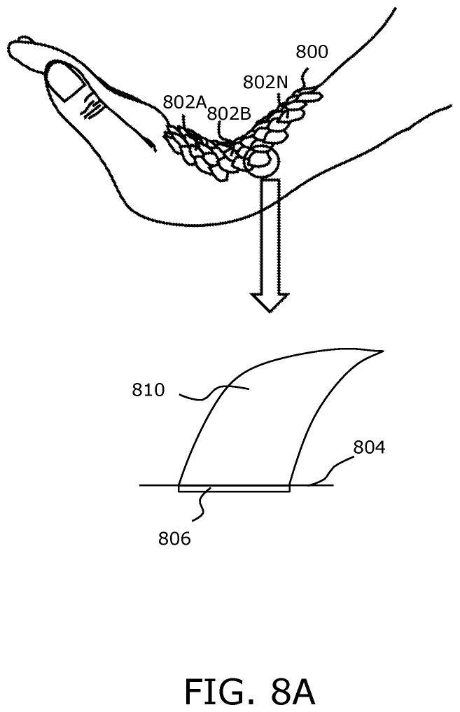

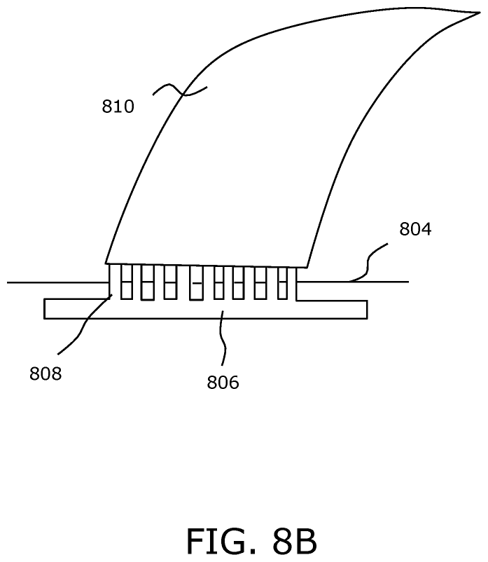

FIG. 8 is a top view of an exemplary wearable protective device 800, in accordance with an embodiment of the present disclosure. The exemplary wearable protective device 800 includes a plurality of three dimensional (3D) scales 802A-N and a flexible base layer arrangement 804 that is connecting each of the plurality of 3D scales 802A-N. Each of the plurality of 3D scales 802A-N includes a bottom layer 806, and a top layer 810 that is present on top of the flexible base layer arrangement 204. The plurality of 3D scales 802A-N are overlapping when the exemplary wearable protective device 800 is placed on a planar surface. The plurality of 3D scales 802A-N are intersecting with each other when a limit of a range of motion is applied to the exemplary wearable protective device 800, to provide a mechanical interlocking effect.

FIG. 8B is a side view of a three-dimensional (3D) scale of FIG. 8A that has a plurality of teeth 808 in accordance with an embodiment of the present disclosure. Each of the plurality of 3D scales 802A-N includes the bottom layer 806, the plurality of teeth 808, and the top layer 810 that is present on top of the plurality of teeth 808. The plurality of teeth 808 may penetrate through the flexible base layer arrangement 804.

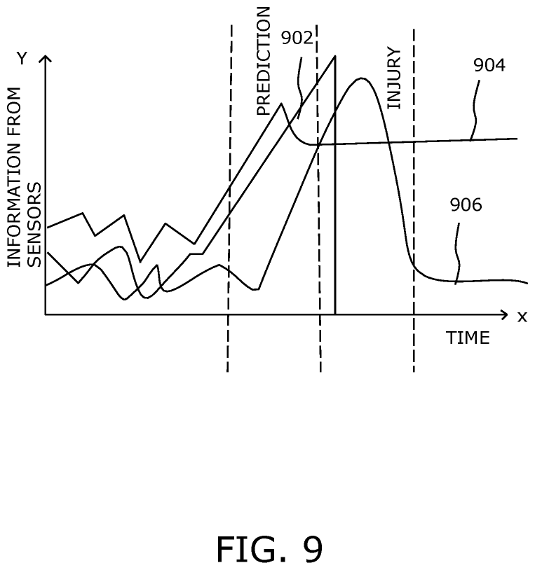

FIG. 9 is an exemplary graphical representation of prediction of an injury based on information retrieved from one or more sensors embedded in a scaled composite structure, in accordance with an embodiment of the present disclosure. In the exemplary graphical representation of the prediction of the injury, in an ordinate Y-axis plotted against time in an abscissa X-axis to information from sensors. A curve 902 represents information retrieved from an accelerator sensor that is embedded in the scaled composite structure, over a period of time. A curve 904 represents information retrieved from a gyroscope sensor that is embedded in the scaled composite structure, over the period of time. A curve 906 represents information retrieved from a flex sensor that is embedded in the scaled composite structure, over the period of time. The exemplary graphical representation shows an injury prediction that is going to happen, for hyperextending wrists based on the information retrieved from one or more sensors embedded in the scaled composite structure.

The one or more sensors in the scaled composite structure may sense the injury if it happens to a body part (example: wrist) and may transfer sensed data to an analytical server to analyse and provide feedback to a user or a wearer to cure the injury.

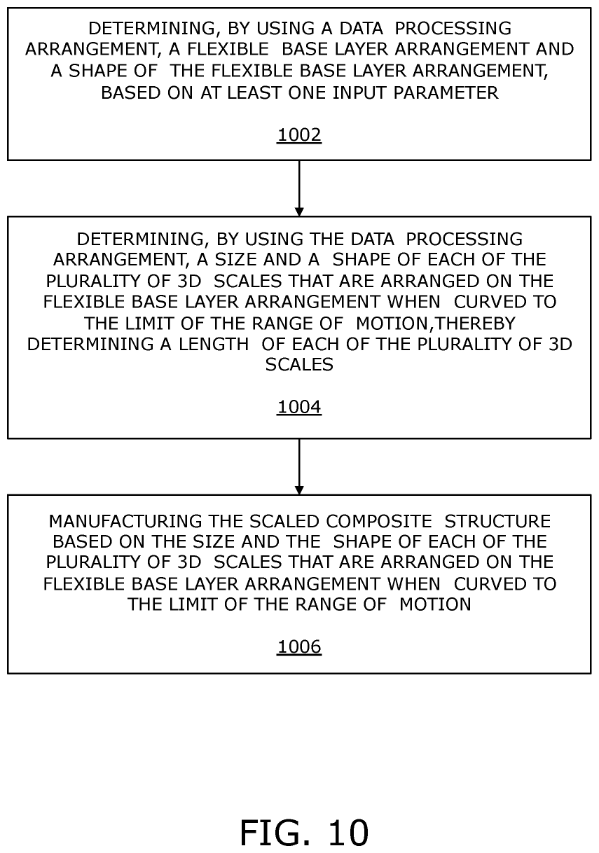

FIG. 10 is a flowchart illustrating steps of a method for designing and manufacturing a scaled composite structure, in accordance with an embodiment of the present disclosure. The scaled composite structure includes a plurality of three-dimensional (3D) scales that are overlapping when the scaled composite structure is placed on a planar surface and a first layer that is connecting each of the plurality of 3D scales. At a step 1002, a flexible base layer arrangement and a shape of a flexible base layer arrangement are determined, using a data processing arrangement, based on at least one input parameter. At a step 1004, a size and a shape of each of the plurality of 3D scales that are arranged on the flexible base layer arrangement when curved to the limit of the range of motion are determined, using the data processing arrangement, thereby determining a length of each of the plurality of 3D scales. At a step 1006, the scaled composite structure is manufactured based on the size and the shape of each of the plurality of 3D scales that are arranged on the flexible base layer arrangement when curved to the limit of the range of motion.

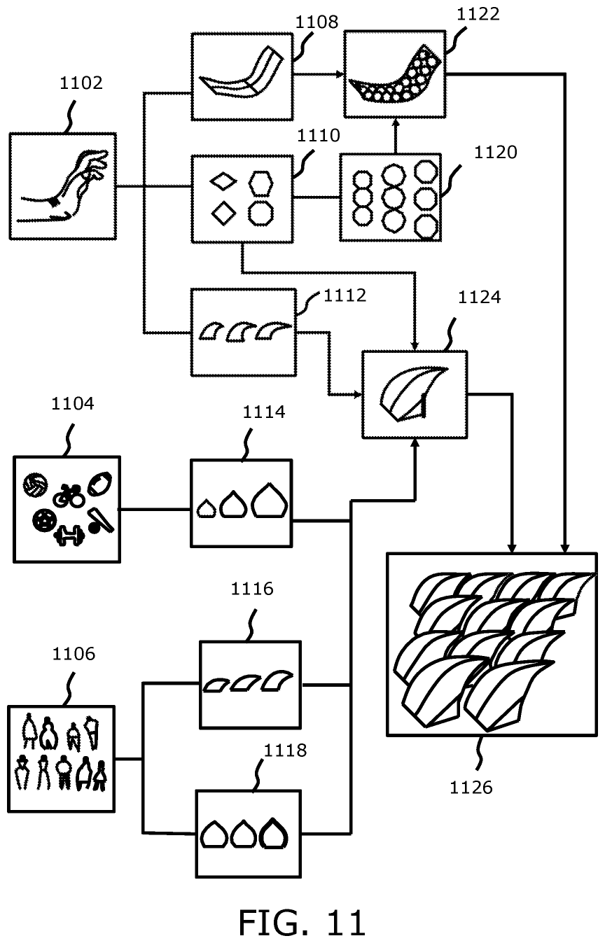

FIG. 11 is a schematic diagram of an exemplary method for designing a scaled composite structure for protecting a body joint, in accordance with an embodiment of the present disclosure. At a step 1102, data associated with a body joint to be protected is received. The data associated with the body joint may include an image of the body joint in a maximum range of motion. Optionally, the data associated with the body joint may include manual input such as a type of the body joint, the maximum range of motion of the body joint, a length and a width of an area to be covered. At a step 1104, a type of activity to be performed by a wearer is received. At a step 1106, body specifications of the wearer are received. At a step 1108, a flexible base layer arrangement is determined based on the data associated with the body joint. At a step 1110, a shape of the flexible base layer arrangement is determined based on the data associated with the body joint. At a step 1112, a length of each of a plurality of 3D scales is determined based on the data associated with the body joint. At a step 1114, a size of each of the plurality of 3D scales is determined based on the type of activity to be performed by the wearer. At a step 1116, a height of each of the plurality of 3D scales is determined based on the body specifications of the wearer. At a step 1118, a thickness of each of the plurality of 3D scales is determined based on the body specifications of the wearer. At a step 1120, a tessellation density is determined based on a shape of a flexible base layer arrangement. At a step 1122, the flexible base layer arrangement when curved to the limit of the range of motion is tessellated into the shape of the base unit based on the tessellation density. At a step 1124, a geometry of each of the plurality of 3D scales is determined based on the length, the size, the height, and the thickness of the each of the plurality of 3D scales. At a step 1126, the scaled composite structure is designed based on tessellated flexible base layer arrangement and the geometry of each of the plurality of 3D scales.

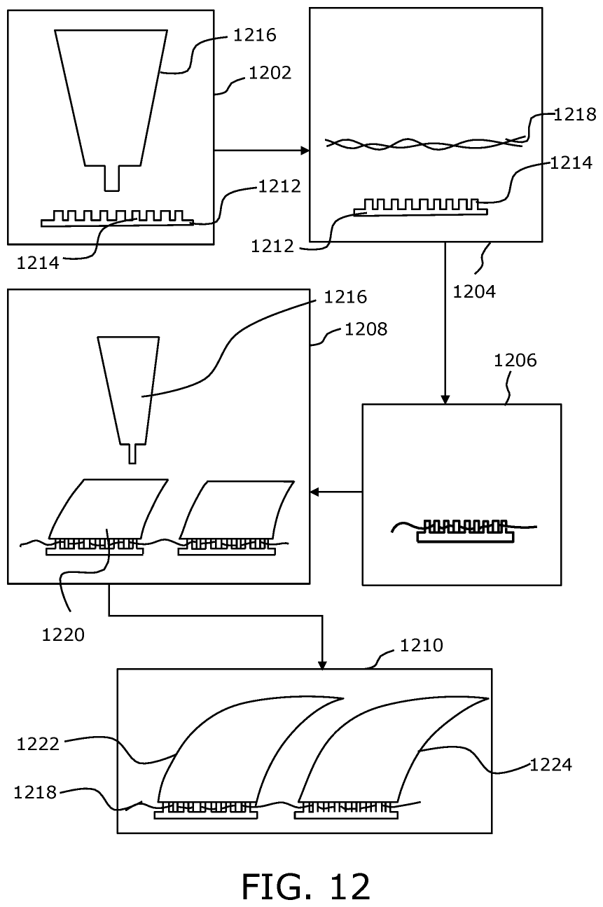

FIG. 12 is a schematic diagram of an exemplary method for manufacturing a scaled composite structure, in accordance with an embodiment of the present disclosure. At a step 1202, a base layer 1212 of a three-dimensional (3D) scale is printed using a 3D printer 1216, thereafter a row of teeth-like structures 1214 is printed on top of the base layer 1212 using the 3D printer 1216. At a step 1204, a first layer 1218 is placed on top of the row of teeth-like structures 1214. At a step 1206, the first layer 1218 is pushed through the plurality of teeth like structures 1214. At a step 1208, a top layer 1220 of the 3D scale is printed, using the 3D printer 1216, on top of the row of teeth-like structures 1214. At a step 1210, a first 3D scale 1222 and a second 3D scale 1224 are manufactured in a row using the 3D printer 1216, that are connected by the first layer 1218.

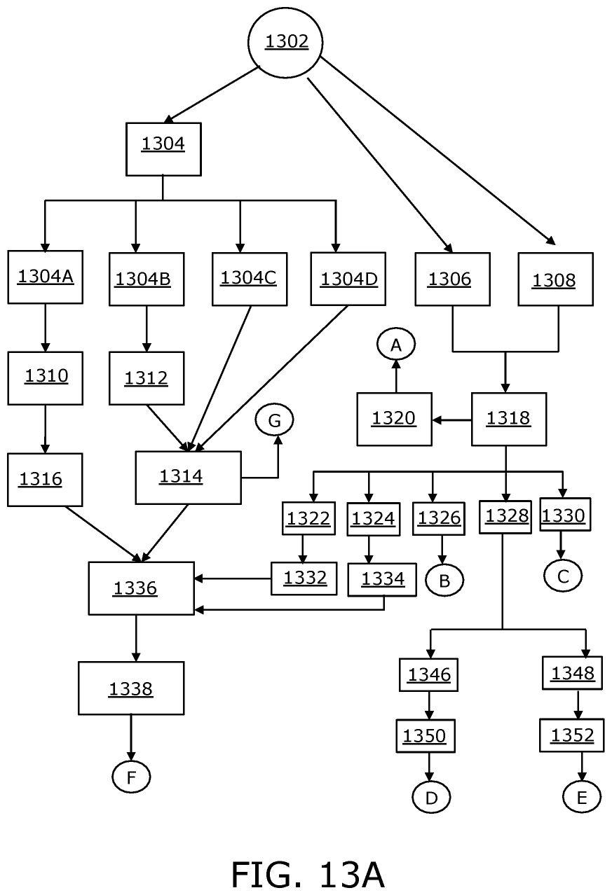

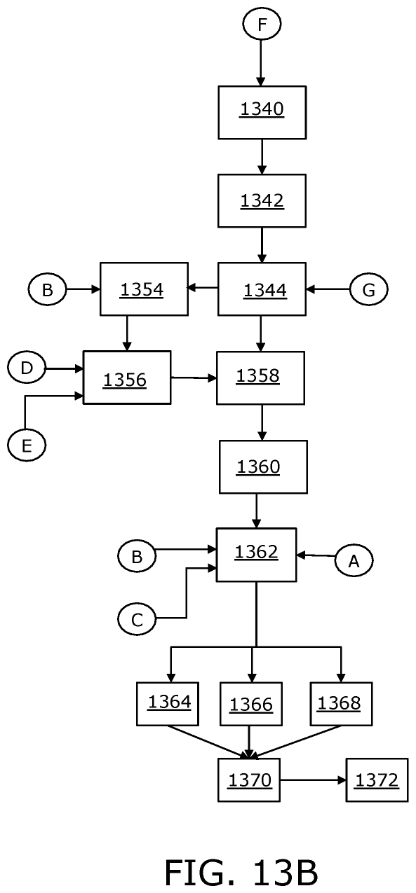

FIGS. 13A-13B are flow charts of an exemplary method for designing and manufacturing a scaled composite structure for protecting a body joint, in accordance with an embodiment of the present disclosure. At a step 1302, a user or a wearer provides at least one input parameter. At a step 1304, data associated with the body joint to be protected is received. The data associated with the body joint to be protected includes a type of body joint 1304A, an image of the body joint in a maximum range of motion or the maximum range of motion of the body joint as a manual input 1304B, a length 1304C of the scaled composite structure needed to cover the body joint, a width 1304D of the scaled composite structure needed to cover the body joint. The maximum range of motion represents a limit of the range of the motion to be applied to the scaled composite structure. At a step 1306, body specifications of the wearer are received. At a step 1308, a type of activity to be performed by the wearer is received. At a step 1310, a range of motion of the body joint is determined based on the type of body joint 1304A. The range of motion of the body joint may be a healthy range of the motion of the body joint. At a step 1312, the maximum range of motion of the body joint is determined based on the image of the body joint in the maximum range of motion or the maximum range of motion of the body joint as the manual input 1304B. At a step 1314, a flexible base layer arrangement to be covered with a plurality of three dimensional (3D) scales is determined based on the maximum range of motion of the body joint, the length 1304C of the scaled composite structure, and the width 1304D of the scaled composite structure. At a step 1316, a shape of a flexible base layer arrangement is determined based on the free range of motion of the body joint. At a step 1318, a force to be applied to the scaled composite structure is estimated based on the type of activity to be performed by the wearer and the body specifications of the wearer. At a step 1320, a total weight of the scaled composite structure is determined based on estimated force to be applied to the scaled composite structure. At a step 1322, a density of the scaled composite structure is determined based on the estimated force to be applied to the scaled composite structure. At a step 1324, a base size of each of the plurality of 3D scales is determined based on the estimated force to be applied to the scaled composite structure. At a step 1326, a thickness of each of the plurality of 3D scales is determined based on the estimated force to be applied to the scaled composite structure. At a step 1328, a type of the flexible base layer arrangement to connect each of the plurality of 3D scales is selected based on the estimated force to be applied to the scaled composite structure. At a step 1330, a type of material to manufacture the plurality of 3D scales is selected based on the estimated force to be applied to the scaled composite structure. At a step 1332, a distance between each of two 3D scales within the scaled composite structure is determined based on the density of the scaled composite structure. At a step 1334, an overall size of each of the plurality of 3D scales is determined based on the base size of each of the plurality of 3D scales. At a step 1336, the flexible base layer arrangement is tessellated into the shape of the base unit based on the overall size of the each of the plurality of 3D scales and the distance between each of the plurality of 3D scales. At a step 1338, each of the plurality of 3D scales is scaled to a size according to the shape of the flexible base layer arrangement and is arranged on the curved base layer according to a tessellation pattern. At a step 1340, an intersection point between each two scales of the plurality of 3D scales in a row of 3D scales disposed on the flexible base layer arrangement when curved to the limit of the range of motion is determined. At a step 1342, a length of each of the plurality of 3D scales in the flexible base layer arrangement is determined based on the intersection point between each two scales of the plurality of 3D scales. At a step 1344, the flexible base layer arrangement is flattened that are arranged with the plurality of 3D scales.

At a step 1346, a degree of fineness of the flexible base layer arrangement is determined based on the type of the flexible base layer arrangement. At a step 1348, a thickness of the flexible base layer arrangement is determined based on the type of the flexible base layer arrangement. At a step 1350, a diameter of a plurality of teeth provided at a surface of the 3D scale facing towards the flexible base layer arrangement is determined based on the degree of fineness of the flexible base layer arrangement. At a step 1352, a height of the plurality of teeth in each of the plurality of 3D scales is determined based on the thickness of the flexible base layer arrangement. At a step 1354, a base layer of each of the plurality of 3D scales is extruded based on the flattened flexible base layer arrangement that is arranged with the plurality of 3D scales and the thickness of each of the plurality of 3D scales. At a step 1356, the plurality of teeth is modelled based on extruded base layer of the each of the plurality of 3D scales, the diameter of the plurality of teeth in each of the plurality of 3D scales, and the height of the plurality of teeth in each of the plurality of 3D scales. At a step 1358, a 3D model of the scaled composite structure is created based on the flattened flexible base layer arrangement that is arranged with the plurality of 3D scales and modelled plurality of teeth. At a step 1360, the 3D model of the scaled composite structure is exported to a slicer of a 3D printer. At a step 1362, the slicer is adjusted based on the 3D model of the scaled composite structure, the thickness of each of the plurality of 3D scales, the type of material to manufacture the plurality of 3D scales and the total weight of the scaled composite structure. At a step 1364, a thickness of an extruder of the 3D printer is adjusted. At a step 1366, a speed of the extruder of the 3D printer is adjusted. At a step 1368, an infill structure and density are adjusted. At a step 1370, a geometric code (GCODE) for 3D printing of the scaled composite structure is developed based on settings of the 3D printer. At a step 1372, the scaled composite structure is manufactured based on developed GCODE.

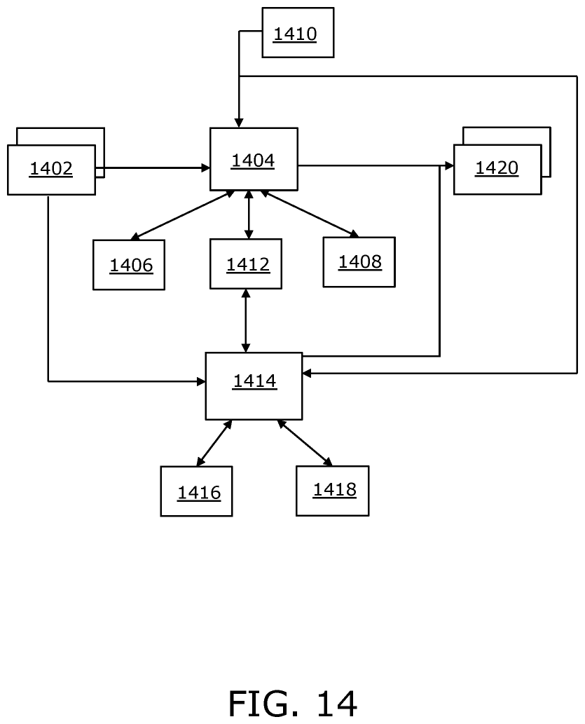

FIG. 14 is an illustration of an exploded view of a distributed computing architecture or a system in accordance with an embodiment of the present disclosure. The exploded view comprises a user device or a client device that comprises an input interface 1402, a control module that comprises a processor 1404, a memory 1406 and a non-volatile storage 1408, processing instructions 1410, a shared or distributed storage 1412, a server that comprises a server processor 1414, a server memory 1416 and a server non-volatile storage 1418 and an output interface 1420. The function of the processor 1404, the memory 1406 and the non-volatile storage 1408 are thus identical to the server processor 1414, the server memory 1416 and the server non-volatile storage 1418 respectively. The functions of these parts are as described above.

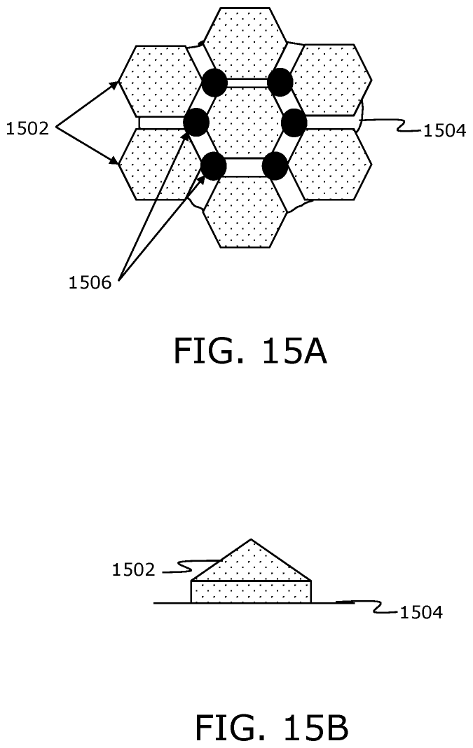

FIGS. 15A and 15B are perspective views of the plurality of 3D scales 1502 connected through a base plate 1504 in accordance with an embodiment of the present disclosure. FIG. shows a top view of the plurality of 3D scales 1502 connected together using the base plate 1504 comprising a plurality of holes 1506. FIG. 15B show a side view of the plurality of 3D scales 1502 connected together using a base plate 1504.

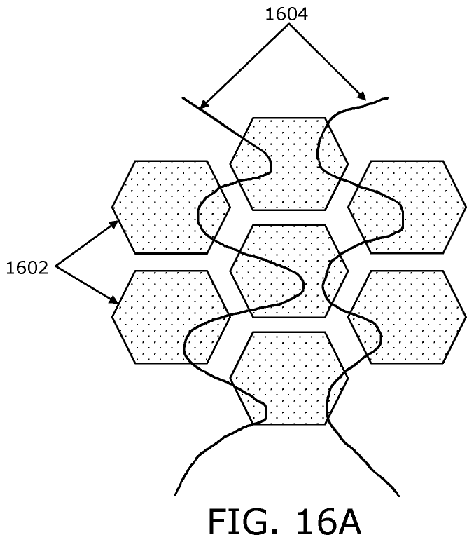

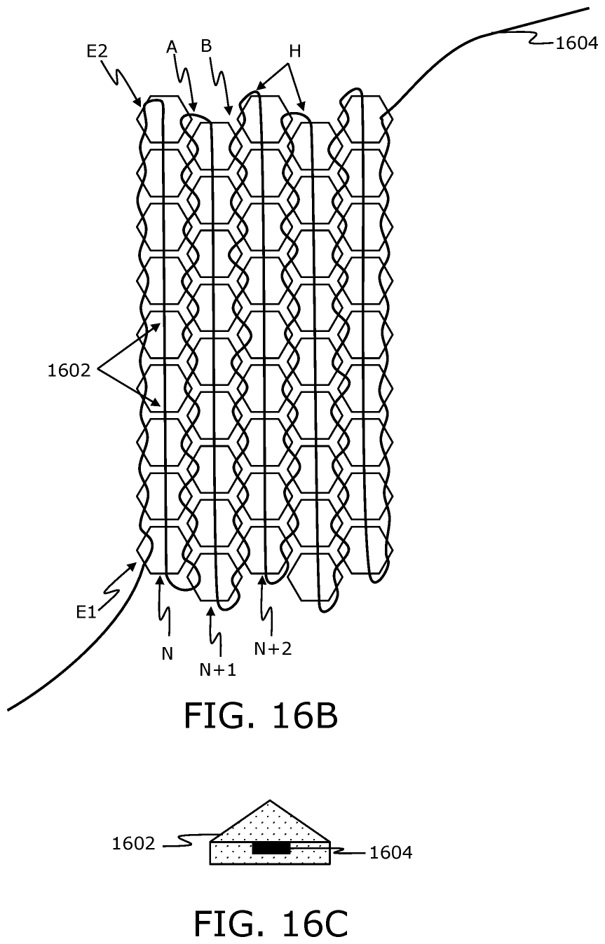

FIGS. 16A, 16B and 16C are perspective views of the plurality of 3D scales 1602 connected through stitches 1604 in accordance with an embodiment of the present disclosure. FIG. 16A shows a top view of the plurality of 3D scales 1602 connected together using the stitches 1604, such as Kevlar stiches. As shown, the Kevlar stitches are used to bond a base unit of a 3D scale 1602 and a top part of an adjacent 3D scale 1602.

FIG. 16B illustrates the plurality of 3D scales 1602 connected through a single length of Kevlar stitch 1604. As shown, in a first stitch A, the Kevlar stitch 1604 connects the adjacent 3D scales 1602, arranged in a N^{th} row and a N^{th}+1 row parallel and adjacent to the N^{th} row, from a first end E1 of each of said parallel rows to a second end E2 of each of said parallel rows. As shown, from the second end E2 of the N^{th}+1 row, the Kevlar stitch 1604 runs centrally through the 3D scales 1602 arranged in the N^{th}+1 row, making a hair-pin loop H like structure at the second end E2 of the N^{th}+1 row.

Similarly, in a second stitch B, the Kevlar stitch 1604 connects the adjacent 3D scales 1602, arranged in N^{th}+1 row and a N^{th}+2 row parallel and adjacent to the N^{th}+1 row, from a first end E1 of each of said parallel rows to a second end E2 of each of said parallel rows. As shown, from the second end E2 of the N^{th}+2 row, the Kevlar stitch 1604 runs centrally through the 3D scales 1602 arranged in the N+2 row, making a hair-pin loop H like structure at the second end E2 of the N^{th}+2 parallel row.

FIG. 16C shows a side view of the plurality of 3D scales 1602 connected together using Kevlar stiches 1604 running in between the adjacent 3D scales 1602.

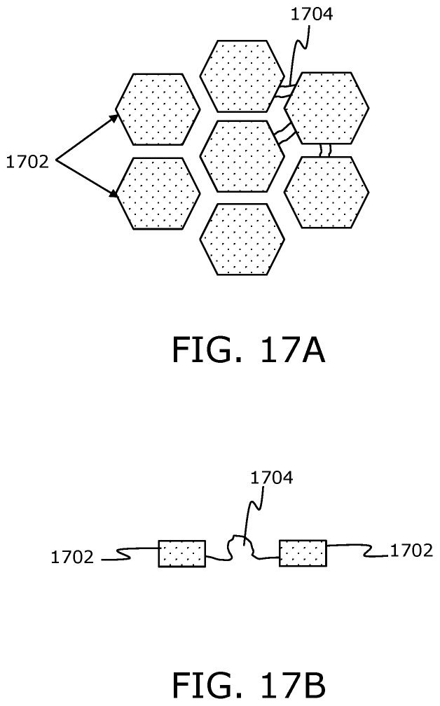

FIGS. 17A and 17B are perspective views of the plurality of 3D scales 1702 connected through living hinges 1704 in accordance with an embodiment of the present disclosure. FIG. 17A shows top view of the plurality of 3D scales 1702 connected together using the living hinges 1704. As shown, connecting the plurality of 3D scales 1702 connected together using the living hinges 1704 does not require a base plate, such as the base plate 1504 of FIG. 15A. FIG. 17B show a side view of the plurality of 3D scales 1702 connected together using living hinges 1704.

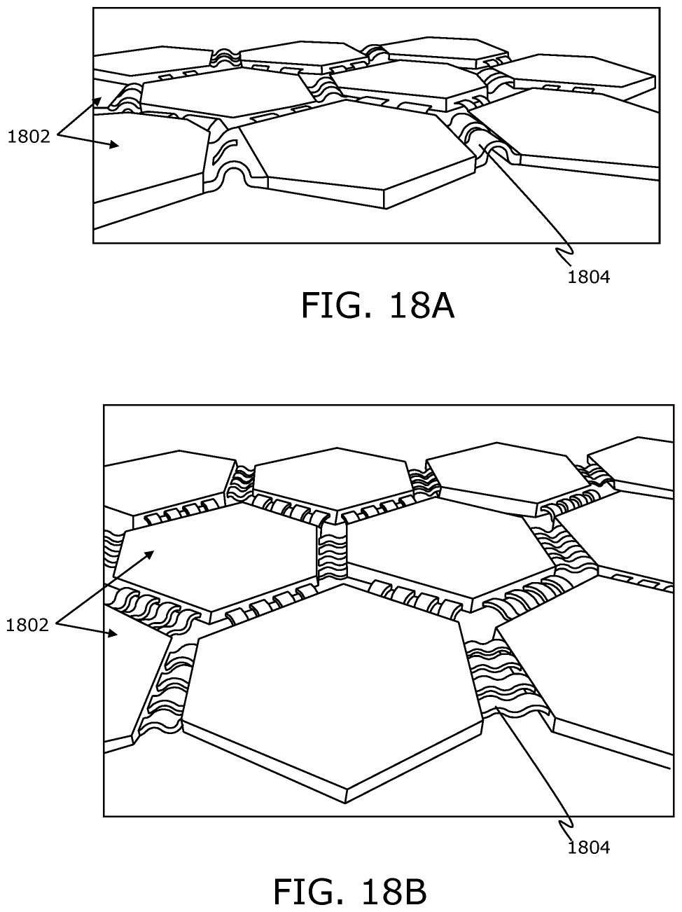

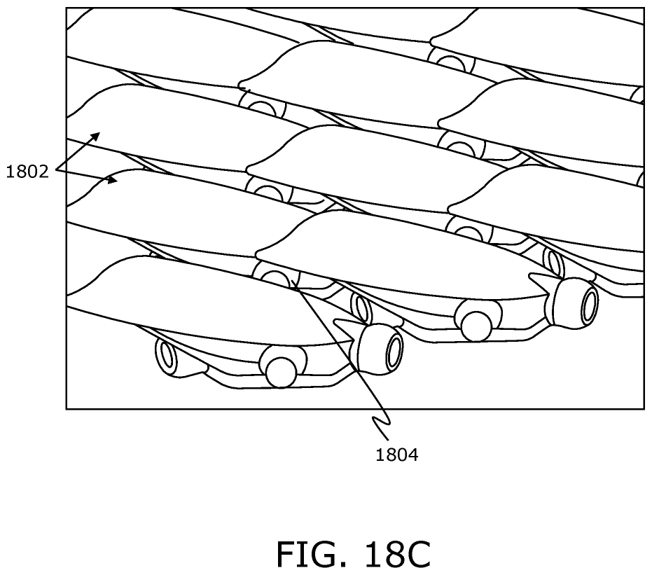

FIGS. 18A, 18B and 18C are illustrations of exemplary living hinges 1802 for connecting plurality of 3D scales 1804 in accordance with an embodiment of the present disclosure. As shown in FIG. 18A, the adjacent 3D scales 1804 are connected together using a pair of living hinges 1802. As shown in FIG. 18B, the adjacent 3D scales 1804 are connected together using four living hinges 1802. As shown in FIG. 18C, the adjacent 3D scales 1804 are connected together using a single living hinge 1802.

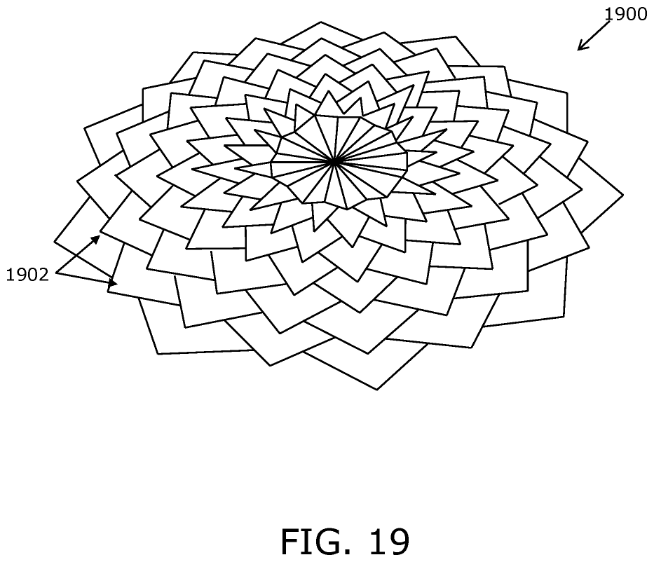

FIG. 19 is an illustration of an exemplary scaled composite structure 1900 in accordance with various embodiments of the present disclosure. As shown, the plurality of 3D scales 1902 are radially arranged to result in a high strength, flexible scaled composite structure 1900.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A scaled composite structure, characterized in that the scaled composite structure comprises:
(i) a flexible base layer arrangement; and
(ii) a plurality of three-dimensional (3D) scales attached to the flexible base layer arrangement, wherein the plurality of 3D scales are overlapping when the scaled composite structure is placed on a planar surface,
wherein a range of motion of the scaled composite structure is controlled through a size and a shape of each of the plurality of 3D scales such that the plurality of 3D scales are intersecting with each other when a limit of the range of motion is applied to the scaled composite structure, to provide a mechanical interlocking effect.

2. The scaled composite structure according to claim 1,
characterized in that the flexible base layer arrangement enables each of the plurality of 3D scales to only rotate around a base plane in a center point of each of the plurality of 3D scales.

3. The scaled composite structure according to claim 1, characterized in that the flexible base layer arrangement operates against a force that is produced when the limit of the range of motion is applied to the scaled composite structure until interlocking of each of the plurality of 3D scales such that the scaled composite structure provides impact protection against produced force through force distribution.

4. The scaled composite structure according to claim 1, characterized in that the size and the shape of at least a given scale of the plurality of 3D scales is a function of a location of the given scale within the scaled composite structure.

5. The scaled composite structure according to claim 1, characterized in that the plurality of 3D scales change color when a force applied over the scaled composite structure exceeds a threshold value.

6. The scaled composite structure according to claim 1, characterized in that a length and a width of the each of the plurality of 3D scales are in a range of 0.01 millimetres (mm) to 500 mm.

7. The scaled composite structure according to claim 1, characterized in that each of the plurality of 3D scales comprises a body portion and a nose portion, wherein the nose portion is characterized as a front extension of each of the plurality of 3D scales which overlaps a preceding 3D scale in the scaled composite structure.

8. The scaled composite structure according to claim 1, characterized in that each scale is attached to the flexible base layer arrangement via a plurality of teeth provided at a surface of the scale facing towards the flexible base layer arrangement, wherein the plurality of teeth are arranged to penetrate through the flexible base layer arrangement to attach to a base plate.

9. A wearable protective device comprises:
(i) a flexible base layer arrangement; and
(ii) a plurality of three-dimensional (3D) scales attached to the flexible base layer arrangement, wherein the plurality of 3D scales are overlapping when the wearable protective device is placed on a planar surface;
wherein a range of motion of the wearable protective device is controlled through a size and a shape of each of the plurality of 3D scales such that the plurality of 3D scales are intersecting with each other when a limit of the range of motion is applied to the wearable protective device, to provide a mechanical interlocking effect.

10. The wearable protective device according to claim 9,
characterized in that the wearable protective device further comprises at least one of a double-sided adhesive layer, a type of hydrogel adhesive layer, a silicone adhesive layer or a rubber adhesive layer on one side of the wearable protective device or a sleeve that is interlaced with the wearable protective device, for attaching to a skin of a wearer.

11. A method for designing and manufacturing a scaled composite structure, wherein the scaled composite structure comprises a plurality of three-dimensional (3D) scales that are attached to a flexible base layer arrangement, wherein the method comprises:
determining, by using a data processing arrangement, a flexible base layer arrangement and a shape of the flexible base layer arrangement, based on at least one input parameter, at a limit of a range of motion to be applied to the scaled composite structure;
determining, by using the data processing arrangement, a size and a shape of each of the plurality of 3D scales that are arranged on the flexible base layer arrangement when curved to the limit of the range of motion, thereby determining a length of each of the plurality of 3D scales; and
manufacturing the scaled composite structure based on the size and the shape of each of the plurality of 3D scales that are arranged on the flexible base layer arrangement when curved to the limit of the range of motion, such that the scaled composite structure provides a mechanical interlocking effect when the limit of the range of motion is applied to the scaled composite structure and is flexible until each of the plurality of 3D scales interlock with each other.

12. The method according to claim 11, further comprises
determining, by using the data processing arrangement, an overall size of each of the plurality of 3D scales and a distance between each of the plurality of 3D scales, based on the at least one input parameter;
tessellating, by using the data processing arrangement, the flexible base layer arrangement when curved to the limit of the range of motion based on the overall size of the each of the plurality of 3D scales and the distance between each of the plurality of 3D scales; and
arranging, by using the data processing arrangement, the plurality of 3D scales on the flexible base layer arrangement according to a size and the shape of a base unit after tessellating the flexible base layer arrangement when curved to the limit of the range of motion to determine the size and the shape of each of the plurality of 3D scales.

13. The method according to claim 11, characterized in that the method further comprises estimating a force to be applied to the scaled composite structure based on the at least one input parameter for enabling determining the overall size of each of the plurality of 3D scales and the distance between each of the plurality of 3D scales.

14. The method according to claim 11, characterized in that the method further comprises determining (i) a thickness of each of the plurality of 3D scales; (ii) a type of the flexible base layer arrangement for connecting each of the plurality of 3D scales; and (iii) a material for manufacturing the plurality of 3D scales, based on an estimated force.

15. The method according to claim 11, characterized in that the method further comprises determining a diameter and a height of a plurality of teeth like structures in each of the plurality of 3D scales based on a degree of fineness and a thickness of the type of the flexible base layer arrangement, wherein the plurality of teeth like structures are configured to penetrate through the flexible base layer arrangement to attach the 3D scales to the flexible base layer arrangement.

16. A The method according to claim 11, characterized in that the method further comprises determining an intersection point between each two scales of the plurality of 3D scales in a row of scales disposed on the flexible base layer arrangement when curved to the limit of the range of motion, to determine the length of the of each of the plurality of 3D scales in the flexible base layer arrangement when curved to the limit of the range of motion.

17. The method according to claim 11, characterized in that the method further comprises generating a 3D model of the scaled composite structure based on a flattened flexible base layer arrangement with the plurality of 3D scales for enabling manufacturing of the scaled composite structure.

18. The method according to claim 11, characterized in that the size and the shape of
at least one a given scale of the plurality of 3D scales are a function of a location of the given scale within the scaled composite structure, wherein the mechanical interlocking effect of the scaled composite structure is controlled through the size and the shape of each of the plurality of 3D scales.

19. The method according to claim 11, characterized in that the manufacturing of the scaled composite structure comprises:
providing a flexible base layer arrangement;
generating a bottom layer of at least one scale of the plurality of 3D scales;
generating a plurality of teeth like structures projecting from the bottom layer;
adding a first layer above the bottom layer; and
generating a top layer of the at least one scale of the plurality of 3D scales on top of the first layer.

20. A computer program product comprising instructions to carry out the method of claim 11.
